(19) United States
(12) Patent Application Publication
(10) Pub. No.: US 2016/0128734 A1
(43) Pub. Date: May 12, 2016

(54) Threaded Setscrew Crosslink

(71) Applicant: Intrepid Orthopedics, Richfield, OH (US)

(72) Inventors: Adam Barlett, Streetsboro, OH (US); Lee Strnad, Richfield, OH (US); Scott Shary, Twinsburg, OH (US)

(21) Appl. No.: 14/937,882

(22) Filed: Nov. 11, 2015

Related U.S. Application Data
(60) Provisional application No. 62/078,340, filed on Nov. 11, 2014.

(51) Int. Cl. A61B 17/70

(52) U.S. Cl. CPC A61B 17/7052; A61B 17/7032; A61B 17/7037; A61B 17/705

(57) ABSTRACT

Devices, systems and methods for connecting surgical screws together, which can be utilized to attach surgical screws in a variety of configurations, such as where the screws and/or attached tulip head components are not parallel or where a significant amount of anatomical variation creates widely divergent screw placement. Also disclosed are low profile crosslink arrangements having a high degree of strength and capable of providing an extremely strong connection to various components of a surgical screw.

### CROSS-REFERENCE TO RELATED APPLICATIONS

This application claims the benefit of U.S. Provisional Patent Application Ser. No. 62/078,340 entitled “Threaded Setscrew Crosslink,” filed Nov. 11, 2014, the disclosure of which is incorporated by reference herein in its entirety.

### TECHNICAL FIELD

The present invention relates to the field of surgery, and more specifically to devices for connecting and maintaining bones or other anatomical structures in a fixed and/or spaced apart relationship, including in conjunction with the use of bone alignment rods and bone screws.

### BACKGROUND OF THE INVENTION

A wide variety of instrumentation systems and surgical techniques have been developed to stabilize and correct spinal conditions and/or deformities, including systems and techniques for correcting degenerative disc disease, spondylolisthesis, spinal deformities, or other spinal conditions through minimally invasive or invasive spinal surgery. In many cases, spinal surgery may include a desire to stabilize a portion of the spine to allow bone or other tissue growth between vertebral bodies, such that a portion of the spine is stabilized or “fused” into a solitary unit and/or specified shape. Commonly known as spinal fusion, this type of stabilization is a commonly-accepted surgical procedure which promotes fusing or growing together of two or more vertebrae in the spine.

The spine is a series of individual bones called vertebrae, separated by cartilaginous disks. The spine includes seven cervical (neck) vertebrae, 12 thoracic (chest) vertebrae, five lumbar (lower back) vertebrae, and the fused vertebrae in the sacrum and coccyx that help to form the hip region. While the shapes of individual vertebrae differ among these regions, each is essentially a short hollow tube containing the bundle of nerves known as the spinal cord. Individual nerves, such as those carrying messages to the arms or legs, enter and exit the spinal cord through gaps between vertebrae. The spinal disks act as shock absorbers, cushioning the spine, and preventing individual bones from contacting each other. Disks also help to hold the vertebrae together. The weight of the upper body is transferred through the spine to the hips and the legs. The spine is held upright through the work of the back muscles, which are attached to the vertebrae. While the normal spine has no significant side-to-side curve, it does have a series of front-to-back curves, giving it a gentle “S” shape. The spine curves in at the lumbar region, back out at the thoracic region, and back in at the cervical region.

One type of spinal fusion procedure is a posterior spinal fusion surgery. This procedure is performed posteriorly, or from the back of patient, as opposed to anteriorly, or through the abdomen. There are many surgical fusion procedures performed with pedicle screw fixation, which can include (among others) posterolateral gutter fusion surgery, posterior lumbar interbody fusion (“PLIF”) surgery and transforaminal lumbar interbody fusion (“TLIF”) surgery. Moreover, there are many approaches and systems for performing posterior spinal surgery. Various exemplary systems can include titanium construction that are compatible with current CT and MRI scanning technology, low profile implant systems, top-loading and top-tightening systems, and other parameters. Some systems also include cross-connectors that allow an implant to be applied across a dual-rod construct for additional strength and stabilization.

A wide variety of popular systems for spinal stabilization and/or fusion employ the use of pedicle or other type screws and rods, in which screw assemblies can be secured into the bony structures of the patient's vertebrae, and one or more rods or other devices are connected between the screw assemblies, typically disposed longitudinally along the length of the spinal segment to anchor vertebral bodies relative to each other. The rods can assume a wide variety of shapes (i.e., straight, curved or irregularly shaped), various positions (i.e., posterior, anterior and/or lateral) and/or configurations (including the use of cross-arms or cross-connections, where desired) according to the patient's anatomy and/or the correction desired. In many cases, the patient's anatomy and/or the desired surgical correction may require aligning one or more rods and associated anchoring screws at numerous different angles and/or orientations along the length of the portion of the treated spinal segment.

Unfortunately, existing pedicle screw systems are typically rather large and bulky, and the modularity and/or flexibility designed into the components in many of these systems can render the systems difficult for a surgeon to use effectively. Because patient anatomy is unique, which can often be compounded by significant preoperative deformity, rarely do the implanted pedicle screw heads conveniently “line up” in a uniform manner. Moreover, assembly difficulties can be experienced when positioning and/or connecting one or more of the rods to the implanted pedicle screws, and when connecting one or more cross connectors to the rods. In many cases, the proper fixation of the stabilization system particularly depends on the surgeon and/or staff to properly assemble the rod and the pedicle system, orient the pedicle screw system, and/or position the rods properly to effectively lock the components together with the set screw.

### SUMMARY OF THE INVENTION

One aspect of the present invention includes a recognition of a need for spinal stabilization systems that incorporate components that can easily accommodate a wide range of patient anatomical variability. Various embodiments described herein are directed to connectors for spinal fixation systems, and more particularly to multi-axial transverse connectors configured to secure multiple pedicle screws. In many embodiments, an object of the present invention is to provide a crosslink assembly which is adjustable in a variety of different ways to give the surgeon options for placement and orientation of the crosslink.

Various of the embodiments described herein relate to devices for connecting a plurality of pedicle screws together to increase torsional rigidity of the system, which can be used in conjunction with a variety of other spinal system components. The variability in the device allows it to attach pedicle screws in a variety of configurations, such as where the screws and/or their tulip head components are not parallel or where a significant amount of anatomical variation creates widely divergent screw placement. The various embodiments also provide a crosslink having a high degree of strength and an extremely strong connection to the tulip head of a pedicle screw, such that a very secure attachment between pedicle screws is provided.

Various embodiments of the present invention will include a multi-axial cross connector that is highly variable and that can desirably include one or more multi-axial ball and socket joints. Various embodiments described herein relate to a transverse cross connector that is configured to connect and maintain a spaced-apart relationship between bone screws and/or other spinal fixation components.

Further features and advantages of the invention, as well as structures and operation of various embodiments of the invention, are further elaborated in detail below with references to the accompanying drawings

### BRIEF DESCRIPTION OF THE DRAWINGS

The present invention should be better understood in conjunction with the detailed description below and the accompanying drawings. In the drawings, like reference numbers typically indicate identical, similar and/or functionally similar elements.

FIG. 1 depicts a front perspective view of an exemplary pedicle screw fixation system incorporating various features of the present invention;

FIG. 2 depicts an exploded partial perspective view of the system of FIG. 1;

FIG. 3 depicts a perspective view of a tulip head component;

FIG. 4 depicts a perspective view of an insert component;

FIGS. 5A through 5E depict various views of one exemplary embodiment of a threaded set screw which incorporates various features of the present invention;

FIGS. 6A and 6B depict perspective and exploded views, respectively, of one exemplary embodiment of a crosslink which incorporates various features of the present invention;

FIGS. 7A through 7D depict various views of one exemplary embodiment of an end set screw which incorporates various features of the present invention;

FIGS. 8A through 8F depict various views of one exemplary embodiment of a crosslink arm which incorporates various features of the present invention;

FIGS. 9A through 9E depict various views of one exemplary embodiment of a housing which incorporates various features of the present invention;

FIGS. 10A through 10D depict various views of one exemplary embodiment of a center set screw arm which incorporates various features of the present invention;

FIGS. 11A through 11D depict various views of one exemplary embodiment of a crosslink, depicting various ranges of component motion and/or anatomical variation that can be accommodated by the construct;

FIGS. 12A and 12B depict perspective views of crosslink embodiments in “housing recessed” and “housing proud” configurations; and

FIGS. 13A and 13B depict perspective views of a crosslink system arranged in two differing configurations to accommodate a similar anatomical distance and screw orientation.

### DETAILED DESCRIPTION OF THE DISCLOSURE

In the following detailed description, for purposes of explanation, numerous specific details are set forth to provide a thorough understanding of the various embodiments of the disclosure. Those of ordinary skill in the art will realize that these various embodiments are illustrative only and are not intended to be limiting in any way. In addition, for clarity purposes, not all of the routine features of the embodiments described herein may be shown or described for every alternative embodiment. One of ordinary skill in the art would readily appreciate that in the development of any such actual implementation, numerous implementation-specific decisions may be required to achieve specific design objectives. These design objectives may vary from one implementation to another and from one developer to another, and the variations thereof are contemplated and included in the present disclosure.

Various of the embodiments described herein include features that facilitate the use and/or modification of surgical constructs, including surgical spinal fusion and/or motion stabilization constructs, which allow the surgeon the ability to accommodate a wide variety of anatomical variation and/or desired surgical correction, yet allows secure fixation of the relevant anatomy when in a tightened or “fixed” condition. In addition, various embodiments described herein facilitate the surgeon's assembly, disassembly and/or adjustment of one or more components intra-operatively.

It should be understood that the term “system,” when referring to various embodiment described in the present invention, can refer to a set of components which includes multiple bone stabilization components such as superior, cephalad or rostral (towards the head) components configured for implantation into a superior vertebra of a vertebral motion segment and inferior or caudal (towards the feet) components configured for implantation into an inferior vertebra of a vertebral motion segment. A pair of such component sets may include one set of components configured for implantation into and for stabilization of the left side of a vertebral segment and another set configured for the implantation into and for stabilization of the right side of a vertebral segment. Where multiple bone segments such as spinal segments or units are being treated, the term “system” may refer to two or more pairs of component sets, i.e., two or more left sets and/or two or more right sets of components. Such a multilevel system can also involve stacking of component sets in which each set includes a superior component, an inferior component, and one or more medial components there between, which may be interconnected and/or independent from each other.

The superior and inferior components (and any medial components there between), when operatively implanted, may be engaged or interface with each other in a manner that enables the treated spinal motion segment to mimic the function and movement of a healthy segment, may alter the relative movement of the various spinal structures in a desired manner and/or may simply fuse the segments such as to eliminate pain and/or promote or enhance healing. The interconnecting or interfacing systems can include one or more structures or members that enable, limit and/or otherwise selectively control spinal or other body motion. The structures may perform such functions by exerting various forces on the system components, and by extension on the target vertebrae. The manner of coupling, interfacing, engagement or interconnection between the subject system components may involve compression, distraction, rotation or torsion, or various combinations thereof. In certain embodiments, the extent or degree of these forces or motions between the components may be intraoperatively selected and/or adjusted to address the condition being treated, to accommodate the particular spinal anatomy into which the system is implanted, and to achieve the desired therapeutic result.

A spinal stabilization system may be installed in a patient to stabilize a portion of a spine, which can include systems that immobilize and/or fixate a specific portion of the spine, as well as systems that control or limit spinal motion to varying degrees (i.e., dynamic stabilization and/or motion limiters). Spinal stabilization may be used, but is not limited to use, in patients having degenerative disc disease, spinal stenosis, spondylolisthesis, pseudoarthrosis, and/or spinal deformities; in patients having fracture or other vertebral trauma; and in patients after tumor resection. A spinal stabilization system may be installed using a minimally invasive procedure. An instrumentation set may include instruments and spinal stabilization system components for forming a spinal stabilization system in a patient.

Crosslink devices such as those described herein can be utilized to facilitate transverse support of the spine in fusion procedures, as well as a variety of other surgical uses. More specifically, embodiments of various crosslink devices described herein may be useful for limiting or eliminating undesired motion (e.g., torsional movement) in a spinal fusion implant. In some applications, variable angle, orientation and/or length crosslink devices can enable a surgeon to extend a fused portion of the spine to additional levels, and/or to augment an existing rod or linkage between levels and/or between pedicles of the same vertebral body. In such cases, the surgeon may use one or more “longer” arm components to bridge extended anatomical spacing, using one or more crosslink devices to provide selective support. The novel crosslink devices may provide several advantages over conventional devices, as persons of ordinary skill in the art who have the benefit of the description of the present disclosure will appreciate.

Components

In various exemplary embodiments, a spinal fusion system (or other orthopedic construct, including spinal motion and/or dynamic stabilization constructs) may contain various combinations, sizes and configurations of the components described hereafter. In an associated exemplary surgical method for implanting a spinal stabilization system, the patient may be placed in a prone position on a surgical table, which for a partially-open and/or minimally-invasive procedure may include a radiolucent table with clearance available for a C-arm of a fluoroscope (i.e., a Jackson table with a radiolucent Wilson frame attachment may be used).

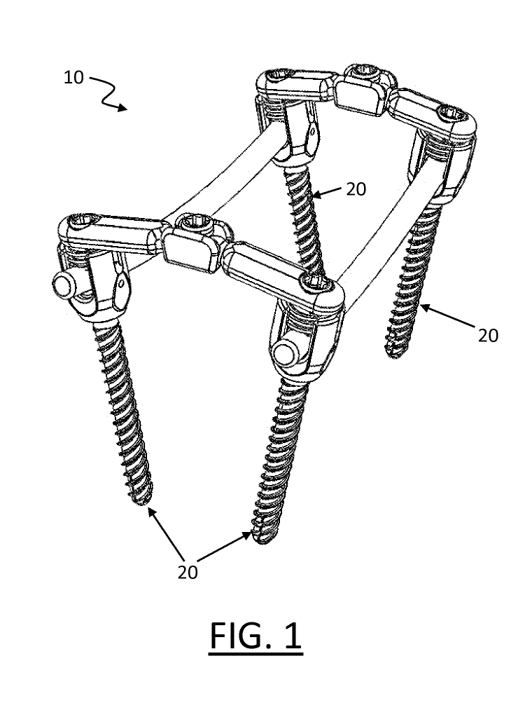

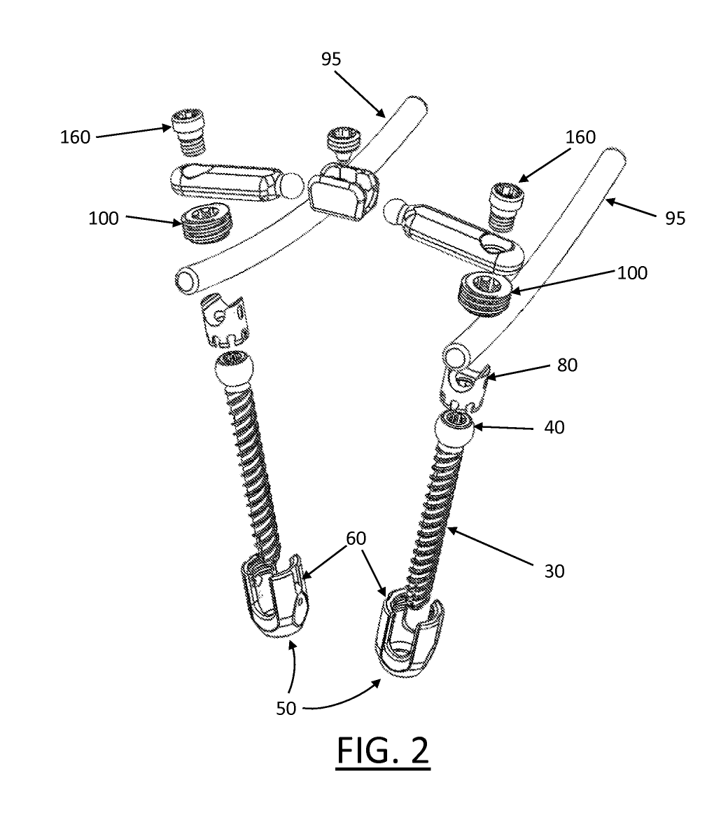

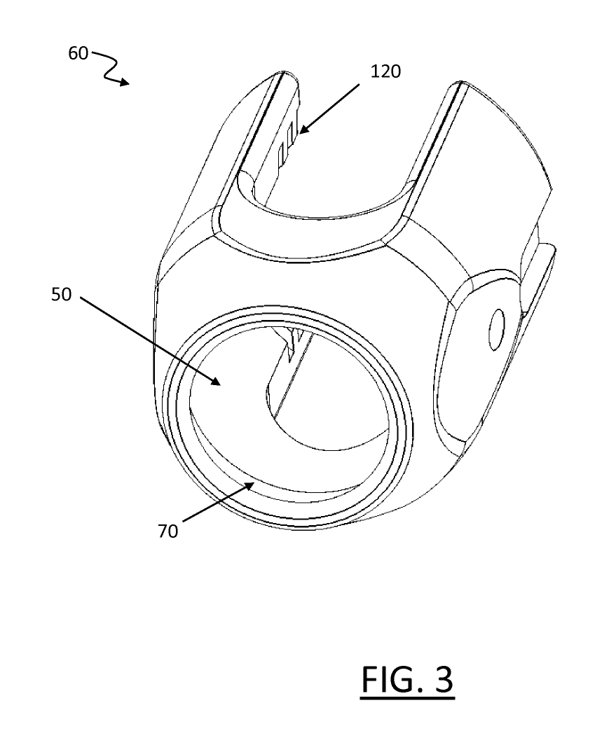

FIG. 1 depicts a front perspective view of one embodiment of a pedicle screw construct 10 in an exemplary “full spinal assembly configuration,” incorporating various features of the present invention. In this embodiment, the system 10 includes pedicle screws 20, which are typically used for securement to bony anatomy (not shown) in a known manner. As can be best seen from FIGS. 2 and 3, each of the pedicle screws 20 can include an adjustable screw shank 30 and a rounded screw head 40, with the screw shank 30 desirably passing through a lower opening 50 in a tulip head connector 60 in a known manner. The adjustability between the rounded screw head 40 and a curved inner surface 70 of the lower opening 50 provides for polyaxial adjustability of the shank 30 relative to the tulip head 60 in a known arrangement. While polyaxial adjustment is described in this embodiment, it should be understood that other types of pedicle screws and/or other attachment devices (i.e., spinal hooks, wire cages, etc.) could be utilized in conjunction with various features of the disclosed invention, including the use of monoaxially adjustable and/or fixed screw shank and connecting tulip head designs.

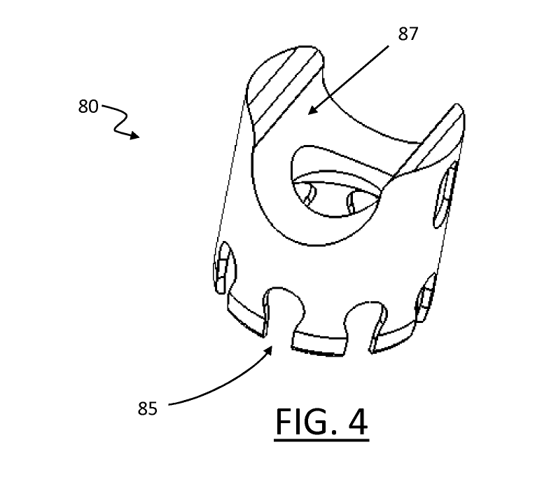

The pedicle screw 20 can further include an insert 80 (see FIG. 4) which desirably fits within the tulip head 60, the insert having a lower insert surface 85 which interacts with and engages the screw head 40 in a known manner. The insert 80 can also have a curved upper surface 87, which desirably engages with a fixation rod 95 or other feature. In the disclosed embodiment, once a fixation rod is positioned within the curved upper surface of the insert, a set screw 100 having an externally threaded surface 110 can desirably be threaded into corresponding internal threads 120 in the tulip head 60, and tightened such that a lower surface of the set screw 100 contacts the rod, which in turn compresses the insert against the screw head and can “lock” or otherwise immobilize the movement of the screw shank relative to the tulip head, as well as to securing the pedicle screw construct and rod together. In the disclosed embodiment, the threaded set screw will desirably engage at least 3 threads with the surround tulip head, although various other construct designs and/or thread forms may desirably engage more and/or fewer threads.

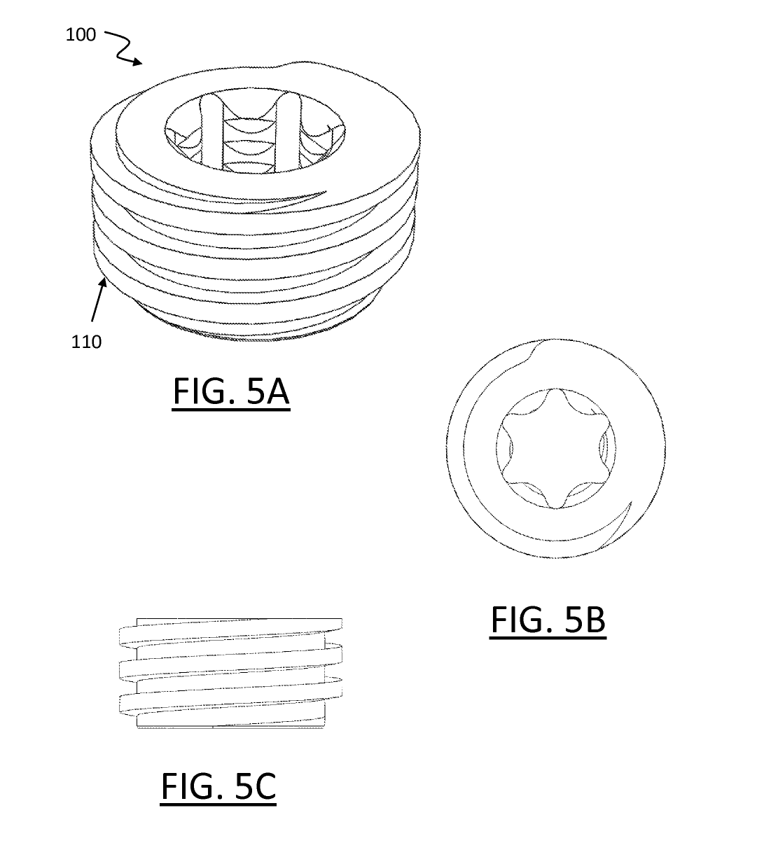

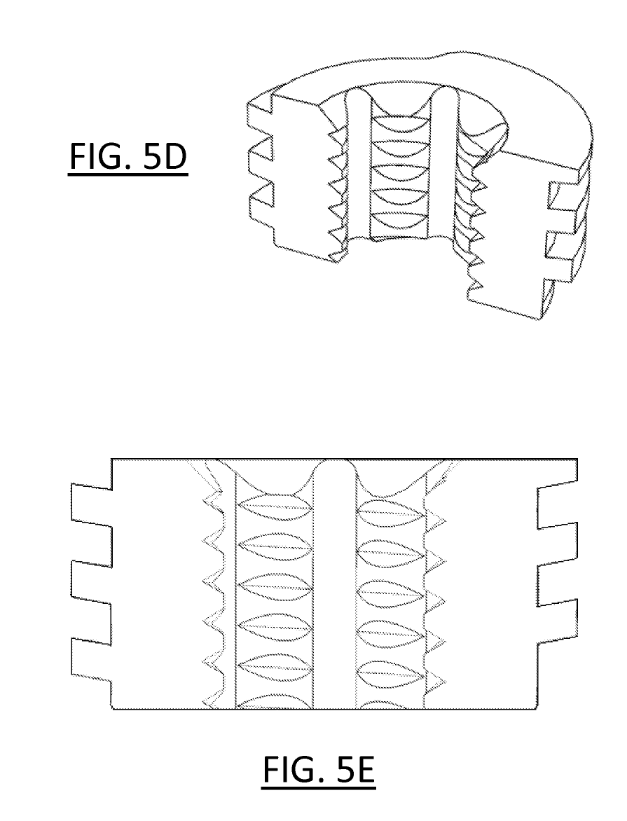

FIGS. 5A through 5E depict various views of one exemplary embodiment of a threaded set screw 100 which incorporates features of the present invention. In this embodiment, the threaded set screw 100 includes an externally threaded surface 110 which desirably engages with corresponding internal threads in a tulip head 60. In one exemplary embodiment, the externally threaded surface 110 includes a sawtooth or other-type thread form having a back angle of approximately 10 degrees, with a corresponding thread form in the interior of the tulip head, which desirably reduces and/or obviates “splay” of the tulip head during tightening of the threaded set screw (although various other thread form shapes and/or dimensions could be utilized for any of the screw threads described herein, as is well known in the art). The threaded set screw 100 further desirably includes an interior bore 120 (which in this embodiment is centrally located, although non-centrally located bores are also contemplated herein) which extends through the threaded set screw 100. The interior bore in this embodiment includes recessed wall sections 130 forming a generally hexalobe shape (which could include the formation of a socket arrangement commonly referred to as a TORX™ socket, commercially available from Camcar Textron of Providence, R.I., USA), which desirably accommodates a hexalobe wrench or other surgical tool for insertion, removal, tightening and/or loosening of the threaded set screw from the tulip head.

In the disclosed embodiment, the various set screws can desirably be tightened using a standard hexalobe-25 screwdriver, with the employment of counter-torque wrenches, as well known in the art, for such tightening actions, if desired. It should be understood that, while a hexalobe shape is depicted in the figure, those skilled in the art should appreciate that the hexalobe configuration described herein, along with the various corresponding surgical tools, could be formed in various alternative shapes, such as a hexagonal socket, a square, a slot, a cross, an oval or other shapes. Similarly, the employment of other size drivers, with various size sockets formed in the various corresponding components. Is contemplated herein. In at least one alternative desired embodiment, the threaded set screw 100 could alternatively incorporate an external camming feature for securement to the tulip head, such as where the internal thread in the tulip head is replace by a recess into which the threaded set screw could lock, as known in the art.

As best shown in the perspective and planar cross-sectional views of the threaded set screw 100 and interior bore 120 of FIGS. 5D and 5E, between each recessed wall section 130 is disposed an inwardly projecting section 140, with each inwardly projecting section also desirably forming an interrupted screw thread or screw thread portions, with the screw thread portions arranged such that the plurality of inwardly projecting sections 140 form a generally threaded interior surface 150 of the interior bore 120. Desirably, the threaded interior surface 150 of the interior bore 120 is sized and configured to accept a corresponding end set screw 160 (see FIG. 2).

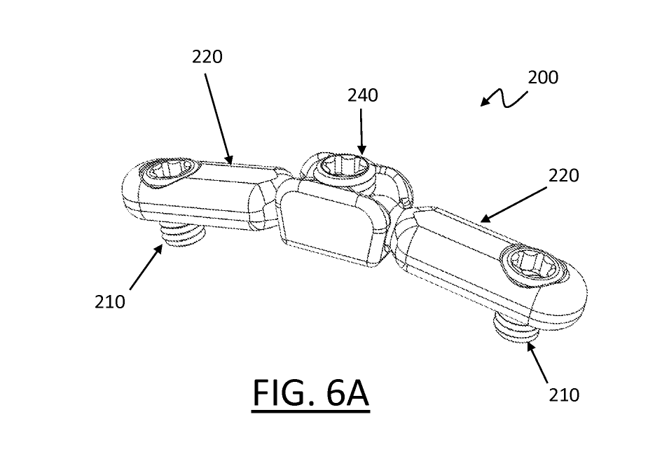

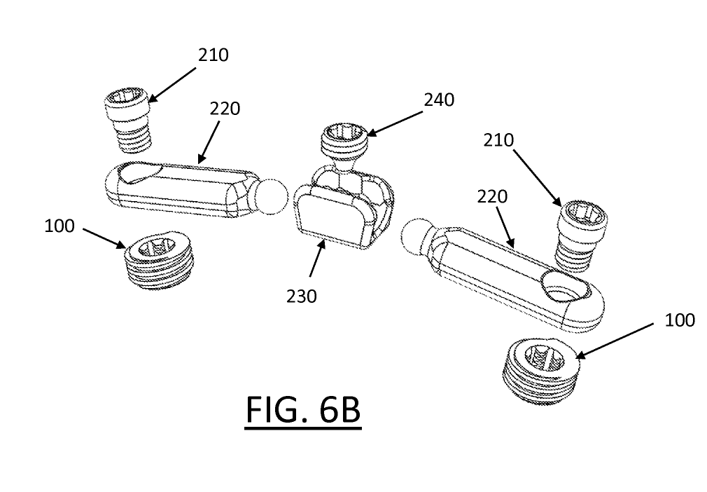

In use, the threaded set screw 100 can desirably be advanced, withdrawn, tightened and/or loosened in a typical manner using an appropriate driving tool (which in this embodiment is a driving tool with a hexalobe engagement end). However, once the threaded set screw is tightened to secure the pedicle screw and rod, the threaded interior surface 150 of the threaded set screw 100 can desirably accommodate a corresponding threaded portion of an end set screw 210, with the threaded set screw 100 utilized as an attachment point for a crosslink 200 (see FIGS. 6A and 6B) or other component. Such an arrangement desirably allows for the various crosslink components to be connected directly to the spinal screws of the spinal construct, rather than attaching to the rods, which can significantly increase the strength and durability of the construct.

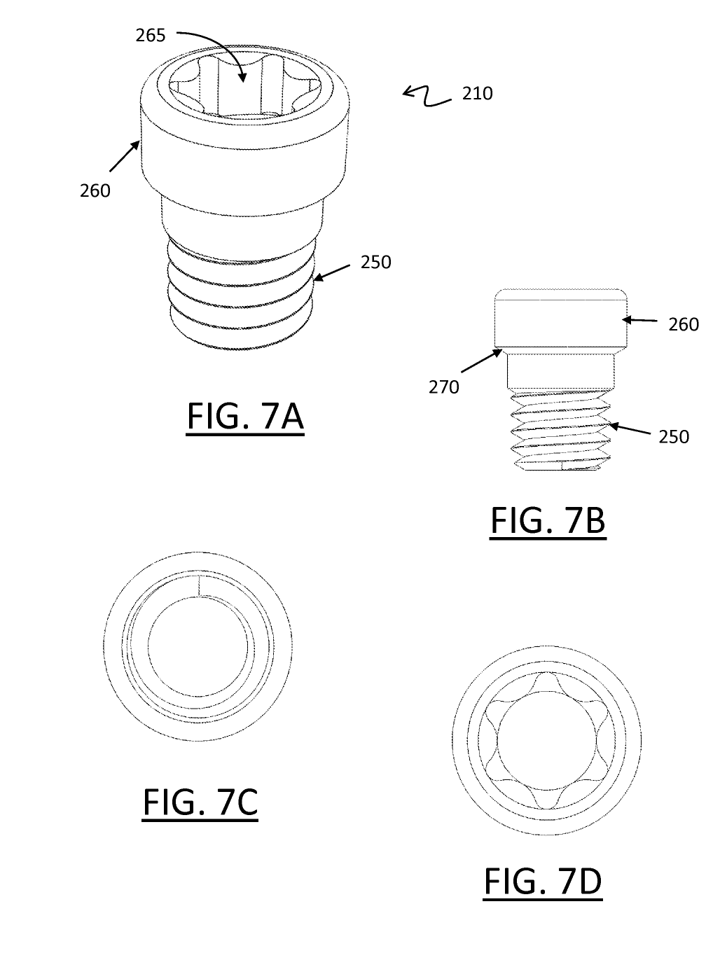

As best seen in FIGS. 6A and 6B, one exemplary embodiment of a crosslink 200 can include a pair of end set screws 210, a pair of arms 220, a housing 230 and a center set screw 240. The end set screws 210 (See FIGS. 7A through 7D) desirably include a threaded shank 250 and an enlarged head portion 260, with a lip 270 formed under the enlarged head portion 260. In the exemplary embodiment, the lip 270 comprises an angled surface of approximately 120 degrees, which can simplify the manufacturability of the lip and the end set screw, although other angles and/or surface features (including rounded and/or recessed lips) could be utilized with varying utility. Moreover, because in various embodiments the thread may not extend fully up to the lip, the lip can allow the end set screw to function in a manner similar to a lag screw, although it should be understood that in other embodiments the thread may extend to and/or into the lip.

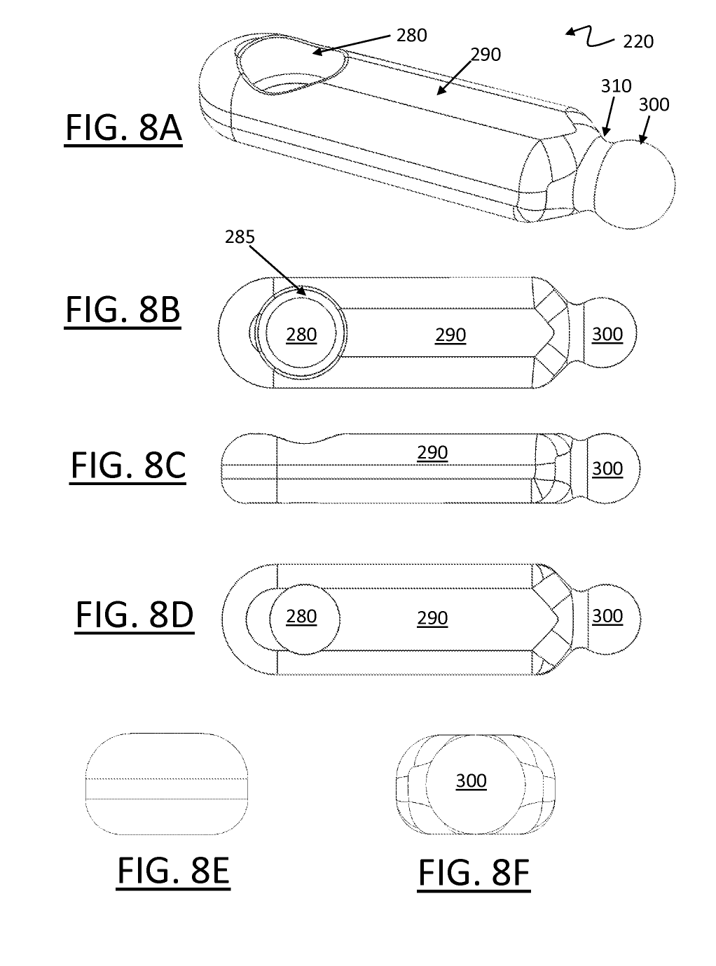

FIGS. 8A through 8F depict one exemplary embodiment of an arm 220. If desired, a surgical kit could include a plurality of arms of differing lengths, diameters, shapes, sizes and/or configurations for use in accommodating a variety of anatomical situations. In this embodiment, the arm 220 includes an arm hole 280, an arm body 290 and a generally spherical arm head 300. The arm hole 280 is desirably sized and configured to accommodate an end set screw 210. A neck portion 310 is formed between the arm body 290 and the arm head 300. While a straight arm is depicted herein, it should be understood that non-straight arms, such as arms having curved and/or angled configurations, could be utilized herein.

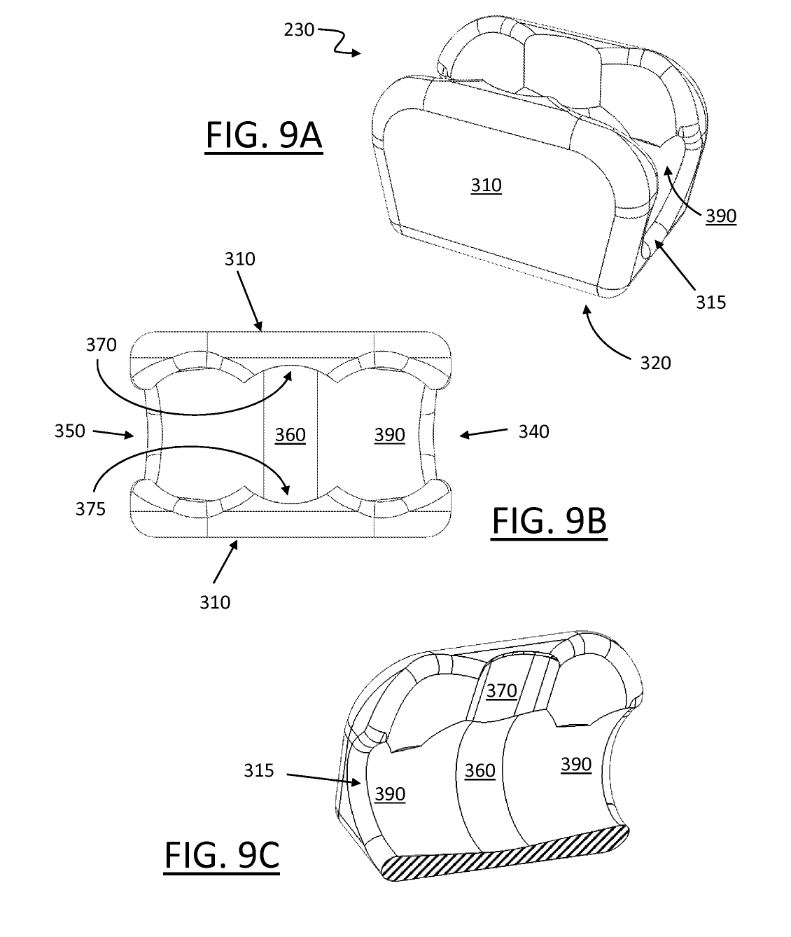

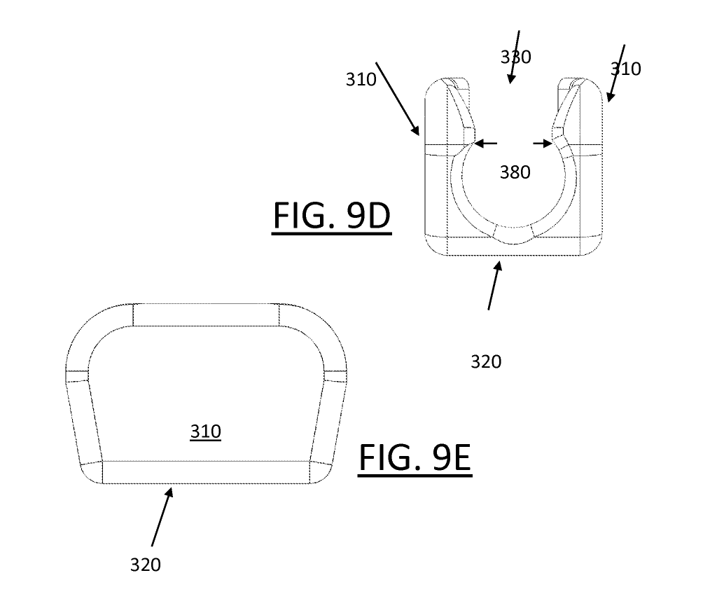

FIGS. 9A through 9E depict one embodiment of a housing 230. The housing desirably mates with the spherical heads of the arms and a center set screw. The housing 230 includes generally opposing sidewalls 310 connected by a lower wall 320, forming an elongated generally U-shape. An opening 330 is formed at the top of the housing 230, with the opening desirably communicating with a medial opening 340 and a lateral opening 350 at the sides of the housing. The housing further includes a central section 360, with inner surfaces 370 and 375 of the housing including generally threaded interior surfaces 380 and 385, which desirably form a threaded receiver in the center section 360 to accommodate a corresponding center set screw 240. In one exemplary embodiment, the generally threaded interior surfaces 380 and 385 could comprise square or “acme-type” threads, although the use of a wide variety of thread forms known in the art for any of the threads and/or threaded components described herein is contemplated herein, including the use of flat threads, negative angle threads and/or saw-tooth threads, such as threads having a 10 degree or other back angle.

While the disclosed housing includes connection points to accommodate a pair of arms, it is conceivable that various alternative embodiments could include a housing having a connection point for only a single arm, such as where the housing could form an integral part of the second arm for connection to the tulip head of the opposing pedicle screw. In such an arrangement it may be desirous to provide additional adjustability at the single connection point (as compared to the adjustability described herein in conjunction with two adjustable arms), which could include the use of a polyaxial and/or monoaxial connection between the housing and the single arm.

In use, each side of the housing will desirably receive and accommodate an arm head 300 of a respective arm 220 (see FIGS. 6A and 6B), which is a generally spherical head on a proximal end of the arm. In use, the spherical arm head 300 can be placed through the top of the housing (i.e., “top-loaded”) through the opening 330, with the neck portion 310 of each arm 220 desirably passing through a respective narrowed section 380 of the housing and the arm head 300 fitting within a rounded receiver portion 390 (i.e., within the generally spherical recess) of the housing 230. Once the arm heads are inserted into the housing, a center set screw 240 can be threaded into the center section 360 of the housing 230 (or this screw may already be positioned in the housing where the crosslink was provided in a preassembled configuration), which can be loosely threaded into the housing to desirably retain the arms 220 within the housing, yet allow the arms 220 some degree of freedom to rotate within the rounded receiver portions 390 relative to the housing 230. The spherical arm heads 300 desirably provide a significant amount of polyaxial movement between the housing and each of the arm heads, allowing for the arms to be placed at a variety of positions, angles and/or orientations in order to achieve a desired orientation of the crosslink and accommodate a wide variety of anatomical variation and/or degree of surgical correction. Desirably, the neck portion 310 comprises an undercut portion that gives additional clearance between the arms and the housing as the arms rotate within the housing. In various embodiment, the outer surface of the housing can include a rounded, notched and/or recessed external surface 315 (i.e., incorporating a recessed or rounded radius around one or more edges of the medial and/or lateral openings) which can provide for additional clearance between the arm and the housing, without significantly compromising the strength and/or integrity of the housing.

If desired, the crosslink 200 can be preassembled with the arms 220 secured within the housing 230, and the center set screw 240 threaded into the housing 230. The preassembled crosslink can be provided to the surgeon in a kit form, which can obviate any need for specialized assembly tools. Because the center set screw need not be tightened at this point, the arms 220 can be moved relative to the housing, if desired, to facilitate attachment of each arm 220 to a respective threaded set screw 100. Once the arms are secured to the threaded set screws, the center set screw 240 can be tightened and the crosslink will assume its desired rigid configuration. If desired, the center set screw may be partially tightened at any point in the procedure (including during the preassembly stage), to create a slight frictional resistance between the arms and the housing, thereby allowing for adjustment of the arms relative to the housing but preventing uncontrolled movement of the arms relative to the housing during placement and implantation, which may be undesirable.

To secure an arm 220 to the desired threaded set screw 100, an end set screw can be employed which mates through the distal arm hole of the arm and threads into the threaded interior surface 150 of the interior bore 120 of the threaded set screw. The end set screw can include a hexalobe socket 265 (or other-type socket, as described herein and known in the art), which in various embodiments can desirably be the same size as a hexalobe socket of the threaded set screw 100 and/or the center set screw 240. The threaded shank 250 of an end set screw 210 can be inserted into and through the arm hole 280 of each arm 220, with the threaded shank 250 extending out of the bottom of the arm 220, and the shank 250 can be threaded into the corresponding threaded interior bore 120. Desirably, the arm hole in the distal end of the arm is sized and configured to mate with various outer surfaces of the end set screw. In this embodiment, the arm hole 280 includes an interior ridge which corresponds to and engages with the lip 270 of the end set screw 210 as the end set screw 210 is advanced into the interior bore 120, which desirably provides a ridged connection between the tulip heads and the crosslink. As the end set screw is fully tightened into the threaded set screw 100, a lower surface of the arm 220 will desirably be drawn into intimate contact with the corresponding upper surface of the threaded set screw 100, securing the arm to the underlying pedicle screw construct in a desired manner. This will desirably create a rigid connection between the arm, the threaded set screw and the tulip head. In various embodiments, the lower end of the end set screw will desirably remain recessed from and/or flush with the bottom surface of the threaded set screw when the end set screw is fully tightened.

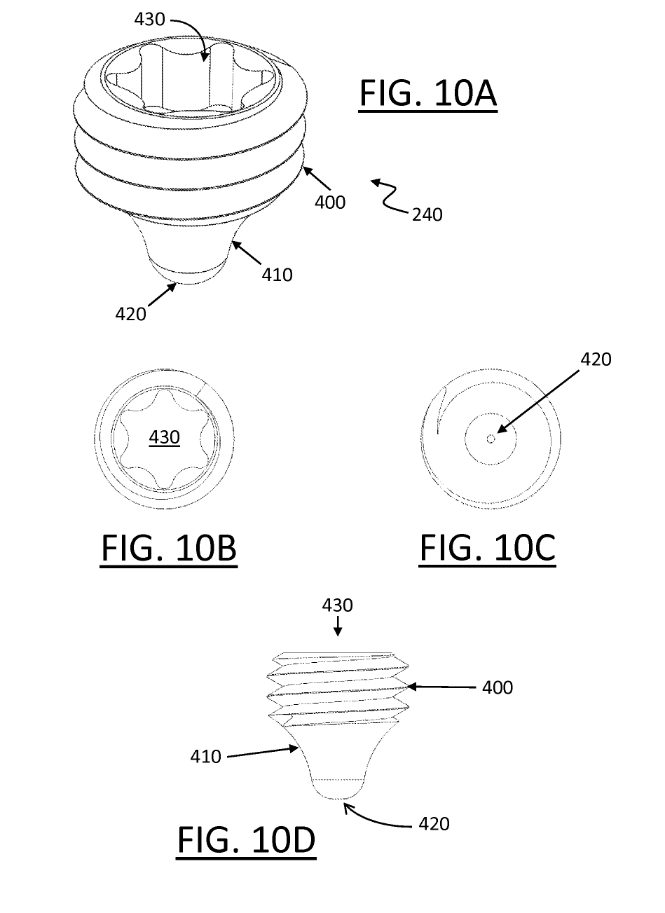

FIGS. 10A through 10D depict various views of a center set screw 240. The center set is threaded into the housing component to retain the arms within the housing, and can eventually be tightened to immobilize the spherical ends of the arms relative to the housing. The center set screw includes a threaded body 400 with a generally conical lower body 410 (of slightly concave shape, in this embodiment) and a rounded lower tip 420. A hexalobe socket 430 is disposed on a top portion of the center set screw 240. In use, the center set screw 240 can be threaded into a corresponding center section 360 of the housing 230, with the conical lower body desirably contacting and wedging the arm heads 300 into contact with the rounded receiver portions 390 of the housing 230. Desirably, further rotation of the center screw will advance the set screw further into the housing, desirably immobilizing the arms 220 within the housing 230.

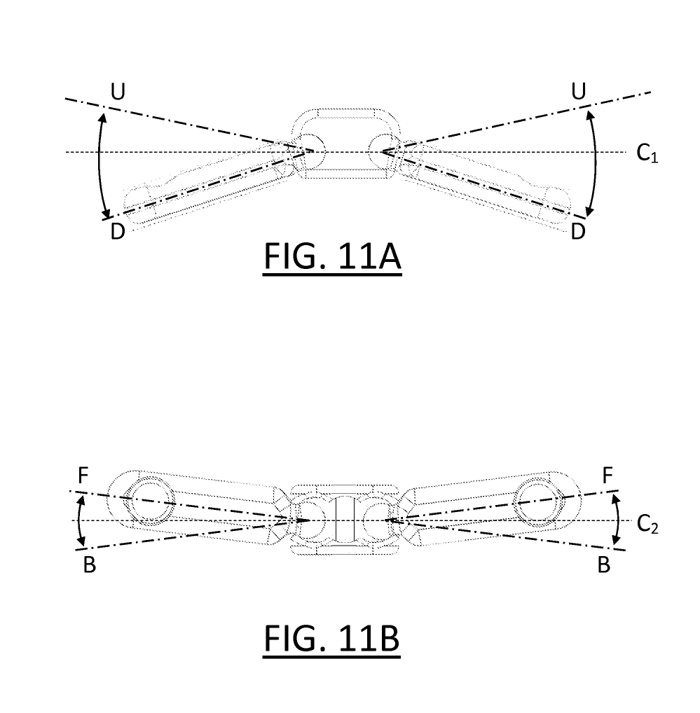

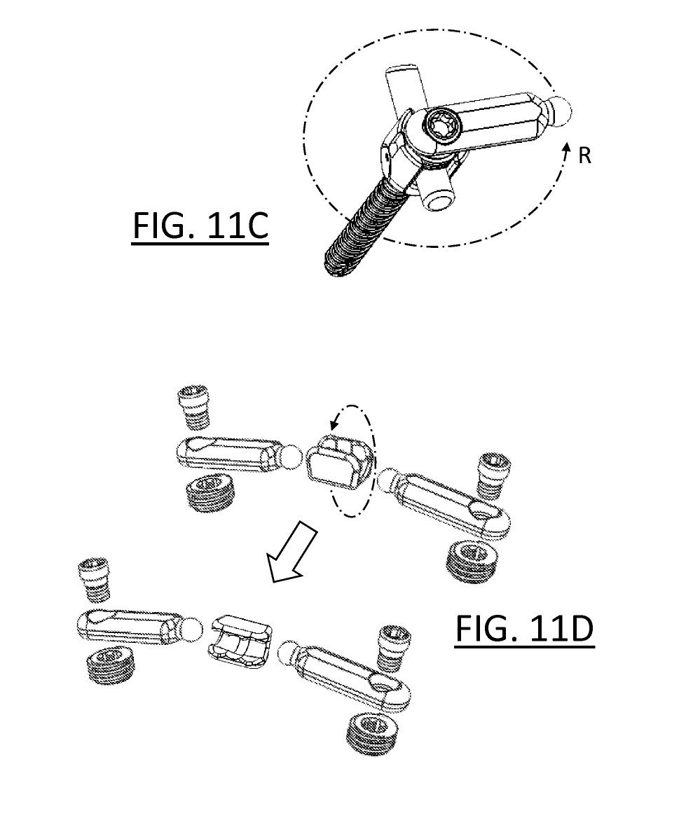

FIGS. 11A through 11C depict various views of one exemplary embodiment of a crosslink, depicting some exemplary ranges of motion and/or anatomical variation that can be accommodated and/or fixated by various components and component linkages of the crosslink system. FIG. 11A depicts some exemplary variation along a first plane accommodated by the linkage between the arms 220 and the housing 230, with the arm heads 300 rotatable within the rounded receiver portions 390 of the housing 230. In this embodiment, each of the arms 220 can be adjusted up to approximately eighteen (18) degrees downward (“D”) from the centerline (“C₁”) of the housing, and up to approximately twelve (12) degrees upward (“U”) from the centerline. FIG. 11B depicts some exemplary variation along a second plane (with the second plane being generally transverse to the first plane in this embodiment) accommodated by the linkage between the arms 220 and the housing 230. In this embodiment, each of the arms 220 can be adjusted up to approximately seven (7) degrees forward (“F”) from the centerline (“C₂”) of the housing, and up to approximately seven (7) degrees backward (“B”) from the centerline. It should also be understood that, if desired, the housing 230 could be rotated relative to the arms to provide further adjustability in a plurality of desired orientations (see FIG. 11D).

FIG. 11C depicts some additional exemplary variation provided by the linkages between each of the arms 220 and the corresponding tulip head 60, which as depicted is facilitated by the connecting mechanism between the end set screw 210 and the threaded set screw 100. In this embodiment, before the end set screw is fully tightened, the arm 220 can be rotated to almost any relative position up to 360 degrees around (“R”) the tulip head. Moreover, the polyaxial adjustability between the screw shank and the tulip head can contribute significant additional variation that can be accommodated by the construct.

In use, a surgeon can initially place the various spinal screws and attached tulip heads into the targeted spinal anatomy of the patient in a known manner, including the use of various preparation and placement tools known in the art for placement of pedicle screws. One or more rods can then be positioned within each of the tulip heads, with the rods locked into each head by a respective threaded set screw. Then one or more crosslinks can be attached to the threaded set screws by end set screws which are introduced through the arm holes and threaded into the threaded set screw of each respective pedicle screw. Desirably, the crosslink will be in an “unlocked” condition at this time, which can allow for relative movement between the housing and arms and a wide range of adjustability to allow the arm holes and end set screws to align with the threaded interior surfaces of the interior bores of the threaded set screws. Once the desired orientation of the arms is achieved, the end set screws can be tightened into the threaded set screws, with the center set screw tightened to lock the housing to the arms and complete immobilization of the spinal construct. If desired, the various connections of the crosslink could be lightly tightened as the construct in placed, and then all connections finally tightened once the construct is in a desired position and orientation.

In the various embodiments, the various screws are desirably secured in their respective final positions by application of a relatively higher torque force to “lock” the screw in a final position in a known manner. However, in various alternative embodiments a locking or camming mechanism could be incorporate into one or all of the screws and/or receiver designs, which could include features to desirably prevent “backing out” of the screw under unusual loading conditions, if desired.

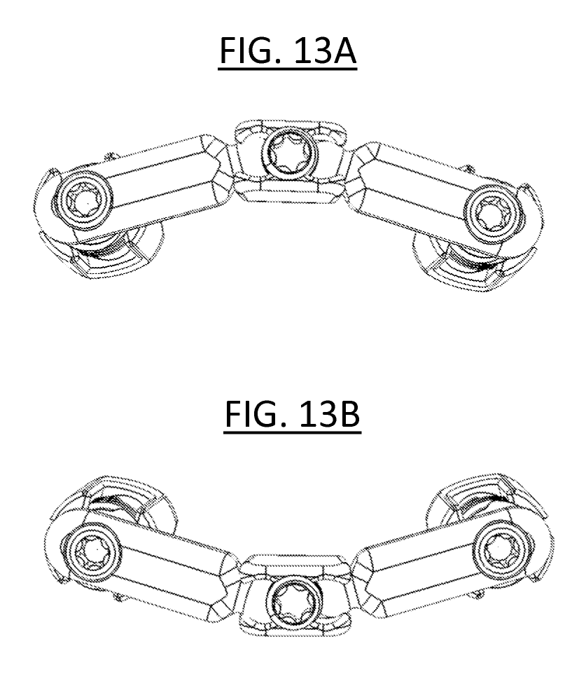

The various features of the disclosed crosslink and spinal construct system represent a significant improvement over preexisting systems in flexibility and versatility. The present components have the capability of being oriented in numerous variations (see FIGS. 13A and 13B for two variants of the same crosslink spanning a similar anatomical distance and screw orientation), as well as allowing for the removal, replacement and/or inclusion of various alternative components, which allows a surgeon the ability to arrange the various components as the surgeon desires. Additionally, it is not necessary to prepare a pre-surgical plan of where the crosslinks must be placed in the patient—rather, the surgeon can simply place the surgical screws and then place a crosslink on such screws as deemed necessary (which may not include every screw used in the surgery, at the surgeon's option).

In the disclosed embodiment, the various crosslink components will desirably increase the posterior profile of the system by only a slight amount, such as by adding approximately 5 mm of height to each of the tulip heads. However, the ability to adjust the rotation of the arms relative to the tulip heads and to adjust the arms relative to the housing can significantly reduce any increased profile. Moreover, the ability of the crosslink to adapt to a wide variety of alignments and/or configurations can facilitate the avoidance of intervening anatomy such as osteophytes and/or bony spurs, as well as healthy bone, nerves, vasculature and/or other tissues.

In one exemplary embodiment, a surgical kit can be provided that provides a plurality of arms of differing lengths, diameters, shapes, sizes and/or configurations for use in accommodating various surgical corrections in a variety of anatomical situations. If desired, a crosslink construct could be assembled from a variety of components such that the medial and lateral portions of the construct are mirror images of each other, or the construct could incorporate dissimilar sized and/or shaped medial and lateral components (i.e., different sized or shaped arms on each side of the housing), including different arms, differing arm terminal ends and/or other dissimilar features. By incorporating interchangeable arms of different lengths, a wide range of sizes for the crosslink can be achieved with a limited catalog of part sizes. For example, a surgical kit could contain a crosslink assembly including a series of arms of different lengths, with the construct capable of being assembled in sizes ranging from 30 mm to 60 mm in 5 mm increments, with each size allowing a variation of between +1 mm to −9 mm of its “stated” (i.e., nominal) size.

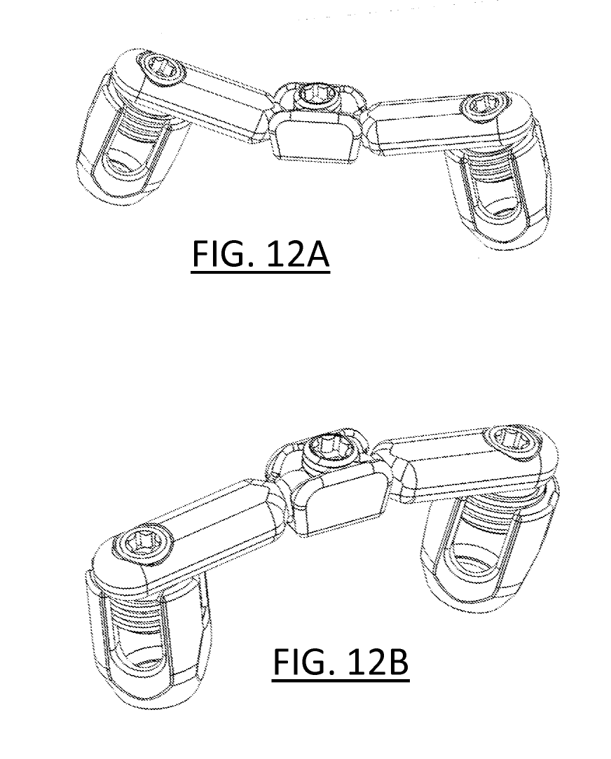

FIGS. 12A and 12B depict another unique aspect of various crosslink embodiments described herein. Because the arms and housing of the crosslink can assume a wide variety of orientations, it would be possible for a first crosslink to assume a “housing recessed” configuration (see FIG. 12A), with a second crosslink assuming a “housing proud” configuration (see FIG. 12B). It is conceivable that such a configuration could facilitate the placement of the first and second crosslinks such that they could potentially cross each other in an “X” configuration, which current cross connector designs cannot accomplish. Moreover, the adjustability and variability attendant in the present systems allow a surgeon to “shape” the crosslink to accommodate a contour of the patient's anatomy, thereby obviating any need to physically contour or “bend” a cross connecting device such as a standard transverse rod in order to fit an implant to a particular patient's anatomy. By conforming to the patient's anatomy, spinal stabilization systems according to the present disclosure may provide better support and immobilization of the spine, and thus may accelerate the healing or fusion processes. This can represent a significant improvement over typical implant components, where a surgeon generally forms elongated members to conform to a patient's anatomy.

Another significant advantage over prior art relates to the simplified connecting and/or fixating mechanisms for the various components described herein as compared to more complex rod clamping and/or rod locking mechanisms of the prior art. Moreover, conventional approaches often involve positioning and fastening a relatively large number of fasteners in order to situate the cross connecting devices as part of the implant, a need that can be obviated by the disclosed designs.

If desired, various embodiments of a crosslink, such as those described herein, could alternatively be utilized to connect a pair of pedicle screws along a longitudinal axis of the patient's spine, such as where a surgeon desired to reinforce a spinal rod along a particularly “stress-prone” portion of the spine, or there was a desired to utilize a crosslink to bridge a bent, broken and/or fractured rod portion, especially where the surgeon might wish to leave portions of the original rod in the patient (i.e., where the rod extend along numerous spinal levels, and the rod at some of those levels is still functional). Where the surgeon wishes to leave such levels undisturbed, the present crosslink and associated components could easily repair the affected level while leaving the remaining spinal levels undisturbed.

Various components of the present invention may also be particularly useful where a surgeon may wish to “retrofit” an existing spinal construct to accommodate one or more crosslinks as described herein. A surgical kit may be provided that contains one or more threaded set screws of an appropriate size to an existing spinal system, along with arms, housings, center set screw and end set screw of appropriate sizes. The surgeon could remove the existing set screw from a tulip head of an existing construct, and introduce the threaded set screw in its place. A crosslink could then be attached to the threaded set screw as described herein.

In various alternative embodiments, a housing could be provided that accommodates more than two arms, such as a housing that accommodates three or four or more arms in a single housing. Such a design could include fixation using a single, centrally located center set screw, or could incorporate multiple set screws with each set screw fixating one or more arms.

In various embodiments, the fixation between each of the arms and the corresponding tulip head (described in various disclosed embodiments) can significantly reduce and/or alter various loads experienced by the lockable connections between the screw shank, the insert and the tulip head of the polyaxial screw. One potential failure mode for currently available polyaxial screw designs can result from external forces attempting to “rotate” the tulip head about the rod and/or about the head of the screw shank. In the present invention, however, the larger moment arm provided by the direct connection between an arm and a corresponding tulip head can provide direct resist to such rotational forces, greatly reducing the opportunity for implant failure. In various embodiments, this feature could significant increase the strength and/or durability of the construct by greatly increasing torsional stability, while in other embodiments such features could permit a redesign of the various screw connections due to the lower and/or altered anticipated loading of the screw.

While the disclosed embodiments describe the crosslink attached to a threaded set screw at each end of the construct, it should be understood that a hybrid crosslink could be provided which connects at one end to a threaded set screw and tulip head, while the opposing end attaches to a rod or other component using a clamp or other known attachment mechanism. If desired, arms of various configurations could be provided in a kit, with various arm components including a variety of terminal ends, including an arm with a threaded set screw attachment, an arm having a rod attachment, an arm having a hook attachment, an arm having a clamp attachment, an arm having a cerclage wire attachment, and/or any combination(s) thereof.

In the various embodiments described herein, the various mating surfaces, adjustable linkages and/or articulating connections could include a variety of frictional and/or engaging features, such as texturing on one or more of the mating surfaces, as well as the use of splines or serrated surfaces between such surfaces. The employment of texturing or other “roughening” of such mating surfaces can significantly increase the strength of the “locked” connection between such surfaces when the various components are tightened, and thereby reduce the opportunity for slippage and/or failure of the one or more linkages under use. For example, in the disclosed system, the various components may include surface texturing of one or more of the engaging surfaces between the housing and the arm, between the center set screw and the arms, between the arm hole and the end set screw, between the arm and the threaded set screw, between the arm and the tulip head, and/or between any of two or more surfaces that desirably engage when the construct is fully tightened.

In conjunction with the various stabilization system components described herein, various surgical instruments may be used in a spinal surgical procedure, including open, partially-open and/or minimally invasive procedures to implant and/or form a spinal stabilization system in a patient. Such instruments can include, but are not limited to, positioning needles, guide wires, dilators, bone awls, bone taps, sleeves, drivers, tissue wedges, trialing and length estimating tools, mallets, tissue retractors, positioning tools and tissue dilators. The instruments may be provided in an instrumentation set. The instrumentation set may also include components of the spinal stabilization system. The components of the spinal stabilization system may include, but are not limited to, bone fastener assemblies of various sizes and/or lengths, elongated members, and closure members.

The various components of the spinal stabilization systems and surgical instruments described herein may be made of a variety of materials including, but not limited to, titanium, titanium alloys, stainless steel, ceramics, and/or polymers. Some components of a spinal stabilization system may be autoclaved and/or chemically sterilized, while others may comprise sterile materials.

All references, including publications, patent applications, and patents, cited herein are hereby incorporated by reference to the same extent as if each reference were individually and specifically indicated to be incorporated by reference and were set Forth in its entirety herein.

The various headings and titles used herein are for the convenience of the reader, and should not be construed to limit or constrain any of the features or disclosures thereunder to a specific embodiment or embodiments. It should be understood that various exemplary embodiments could incorporate numerous combinations of the various advantages and, or features described, all manner of combinations of which are contemplated and expressly incorporated hereunder.

The use of the terms “a” and “an” and “the” and similar referents in the context of describing the invention are to be construed to cover both the singular and the plural, unless otherwise indicated herein or clearly contradicted by context. The terms “having,” “including,” and “containing” are to be construed as open-ended terms (i.e., meaning “including, but not to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., i.e., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A bone anchor assembly, comprising
a first fixation element configured to couple to a bone, the fixation element having a shank portion and a head portion, the shank portion having an external thread form for engaging bone;
a housing portion having an internal bore extending therethrough, the housing portion having at least two sidewalls forming a first channel adapted to receive a connecting rod, at least a portion of each sidewall including an internally facing thread form; and
a screw for placement at least partially within an internal bore of the housing portion, the screw having an external thread form adapted for engagement with the internally facing thread form of the sidewalls, the screw having a top surface with a recess formed therein, the recess including a driver engagement portion formed into a plurality of walls of the recess for engaging a driving tool, the recess further including a threaded section formed into the plurality of walls of the recess, the threaded section at least partially overlapping the driver engagement portion.

2. The bone anchor assembly of claim 1, wherein the driver engagement portion comprises a hexalobular socket.

3. The bone anchor assembly of claim 1, wherein the driver engagement portion comprises a hexagonal socket.

4. The bone anchor assembly of claim 1, further comprising an end set screw having an external thread form adapted for engagement with the threaded section of the recess.

5. The bone anchor assembly of claim 4, further comprising an arm having an arm hole, the arm hole sized to accommodate the end set screw.

6. The bone anchor assembly of claim 5, wherein the arm has a proximal end and a distal end, with the arm hole of the arm positioned proximate the proximal end of the arm and a generally spherical arm head positioned proximate to the distal end of the arm.

7. The bone anchor assembly of claim 6, further comprising a generally u-shaped housing having an interior bore, the interior bore having an internally threaded central portion and an adjacent concave portion for accommodating the spherical arm head of the arm.

8. A polyaxial pedicle screw system comprising:
an anchoring member having a threaded shank and a spherical connector;
a connecting assembly having a socket for receipt of the spherical connector and an opening for receipt of a connecting rod member; and
a fastener securable to an upper portion of the connecting assembly, the fastener including a lower surface for engaging a surface of the connecting rod member and an upper surface having a socket shaped for engaging a driving tool, said socket further including an internally threaded portion.

9. The polyaxial pedicle screw system of claim 8, wherein the socket comprises a hexalobular shape.

10. The polyaxial pedicle screw system of claim 8, wherein the socket comprises a hexagonal shape.

11. The polyaxial pedicle screw system of claim 8, wherein the socket comprises a square shape

12. The polyaxial pedicle screw system of claim 8, wherein the fastener comprises a set screw.

13. The polyaxial pedicle screw system of claim 8, wherein the fastener further includes an externally threaded outer surface.

14. The polyaxial pedicle screw system of claim 8, wherein the opening is U-shaped for receipt of the connecting rod member.

15. The polyaxial pedicle screw system of claim 8, wherein the socket is adapted to accommodate a non-threaded driving tool.

16. A surgical screw assembly comprising:
a central body having a downwardly extending shank, the shank including an externally threaded shaft;
an upwardly facing recess formed in an upper surface of the central body, the upwardly facing recess including a driver engagement portion formed into the peripheral walls of the recess for engaging a driving tool, the upwardly facing recess further including an internal thread form formed into at least a portion of the peripheral walls of the recess.

17. The surgical screw assembly of claim 16, wherein the downwardly extending shank is polyaxially adjustable relative to the central body.

18. The surgical screw assembly of claim 16, wherein the downwardly extending shank is monoaxially adjustable relative to the central body.

19. The surgical screw assembly of claim 16, wherein the upper surface of the central body comprises a removable set screw.

20. The surgical screw assembly of claim 16, wherein the shank further includes a shank head, the central body includes a screw hole, and the screw hole has a smaller inner diameter than an outer diameter of the shank head.
